(19) United States
(12) Patent Application Publication
(10) Pub. No.: US 2009/0111822 A1
(43) Pub. Date: Apr. 30, 2009

(54) 1,2,3-TRIAZOLE PYRROLIDINE DERIVATIVES AS MODULATORS OF MGLUR5

(75) Inventors: Emma BRATT, Molndal (SE); Kenneth Granberg, Molndal (SE)

Correspondence Address: BIRCH, STEWART, KOLASCH & BIRCH, LLP, P.O. BOX 747, FALLS CHURCH, VA, 22040-0747, US

(73) Assignee: AstraZeneca AB, Sodertalje (SE)

(21) Appl. No.: 12/258,161

(22) Filed: Oct. 24, 2008

Related U.S. Application Data
(60) Provisional application No. 60/982,954, filed on Oct. 26, 2007.

(51) Int. Cl. A61K 31/501; C07D 249/08; C07D 403/14; A61K 31/497; A61P 1/04; A61P 25/22; C07D 401/14; A61K 31/4196

(52) U.S. Cl. 514/252.05; 548/266.2; 544/238; 544/333; 544/405; 514/383; 514/255.05; 514/256

(57) ABSTRACT

The present invention is directed to novel compounds, to a process for their preparation, their use in therapy and pharmaceutical compositions comprising the novel compounds.

### FIELD OF THE INVENTION

The present invention is directed to novel compounds, their use in therapy and pharmaceutical compositions comprising said novel compounds.

### BACKGROUND OF THE INVENTION

Glutamate is the major excitatory neurotransmitter in the mammalian central nervous system (CNS). Glutamate produces its effects on central neurons by binding to and thereby activating cell surface receptors. These receptors have been divided into two major classes, the ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels. Schoepp et at., Trends Pharmacol Set. 14:13 (1993), Schoepp, Neurochem. Int. 24:439 (1994), Pin et al., Neuropharmacology 34:1 (1995), Bordi and Ugolini, Prog. Neurobiol. 59:55 (1999).

Molecular cloning has identified eight distinct mGluR subtypes, termed mGluR1 through mGluR8. Nakanishi, Neuron 13:1031 (1994), Pin et al., Neuropharmacology 34:1 (1995), Knopfel et al., J. Med. Chem. 38:1417 (1995). Further receptor diversity occurs via expression of alternatively spliced forms of certain mGluR subtypes. Pin et al., PNAS 89:10331 (1992), Minakami et al., BBRC 199:1136 (1994), Joly et al., J. Neurosci. 15:3970 (1995).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR1, mGluR5 and their alternatively spliced variants. The binding of agonists to these receptors results in the activation of phospholipase C and the subsequent mobilization of intracellular calcium.

### Neurological, Psychiatric and Pain Disorders

Attempts at elucidating the physiological roles of Group I mGluRs suggest that activation of these receptors elicits neuronal excitation. Various studies have demonstrated that Group I mGluR agonists can produce postsynaptic excitation upon application to neurons in the hippocampus, cerebral cortex, cerebellum, and thalamus, as well as other CNS regions. Evidence indicates that this excitation is due to direct activation of postsynaptic mGluRs, but it also has been suggested that activation of presynaptic mGluRs occurs, resulting in increased neurotransmitter release. Baskys, Trends Pharmacol. Sci. 15:92 (1992), Schoepp, Neurochem. Int. 24:439 (1994), Pin et al., Neuropharmacology 34:1 (1995), Watkins et al., Trends Pharmacol Sci. 15:33 (1994).

Metabotropic glutamate receptors have been implicated in a number of normal processes in the mammalian CNS. Activation of mGluRs has been shown to be required for induction of hippocampal long-term potentiation and cerebellar long-term depression. Bashir et al., Nature 363:347 (1993), Bortolotto et al., Nature 368:740 (1994), Aiba et al., Cell 79:365 (1994), Aiba et al., Cell 79:377 (1994). A role for mGluR activation in nociception and analgesia also has been demonstrated, Meller et al., Neuroreport 4: 879 (1993), Bordi and Ugolini, Brain Res. 871:223 (1999). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex. Nakanishi, Neuron 13: 1031 (1994), Pin et al., Neuropharmacology 34:1, Knopfel et al., J. Med. Chem. 38:1417 (1995).

Further, Group I metabotropic glutamate receptors and mGluR5 in particular; have been suggested to play roles in a variety of pathophysiological processes and disorders affecting the CNS. These include stroke, head trauma, anoxic and ischemic injuries, hypoglycemia, epilepsy, neurodegenerative disorders such as Alzheimer's disease and pain. Schoepp et al., Trends Pharmacol. Sci. 14:13 (1993), Cunningham et al., Life Sci 54:135 (1994), Hollman et al., Ann. Rev. Neurosci. 17:31 (1994), Pin et al., Neuropharmacology 34:1 (1995), Knopfel et al., J. Med. Chem. 38:1417 (1995), Spooren et al., Trends Pharmacol. Sci. 22:331 (2001), Gasparini et al. Curr. Opin. Pharmacol. 2:43 (2002), Neugebauer Pain 98:1 (2002). Much of the pathology in these conditions is thought to be due to excessive glutamate-induced excitation of CNS neurons. Because Group I mGluRs appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial, specifically as neuroprotective agents, analgesics or anticonvulsants.

Recent advances in the elucidation of the neurophysiological roles of metabotropic glutamate receptors generally and Group I in particular, have established these receptors as promising drug targets in the therapy of acute and chronic neurological and psychiatric disorders and chronic and acute pain disorders.

### Gastrointestinal Disorders

The lower esophageal sphincter (LES) is prone to relaxing intermittently. As a consequence, fluid from the stomach can pass into the esophagus since the mechanical barrier is temporarily lost at such times, an event hereinafter referred to as “reflux”.

Gastro-esophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD.

The novel compounds according to the present invention are assumed to be useful for the inhibition of transient lower esophageal sphincter relaxations (TLESRs) and thus for treatment of gastro-esophageal reflux disorder (GERD).

It is well known that certain compounds may cause undesirable effects on cardiac repolarisation in man, observed as a prolongation of the QT interval on electrocardiograms (ECG). In extreme circumstances, this drug-induced prolongation of the QT interval can lead to a type of cardiac arrhythmia called Torsades de Pointes (TdP; Vandenberg et al. hERG K⁺ channels: friend and foe. Trends Pharmacol Sci 2001; 22: 240-246), leading ultimately to ventricular fibrillation and sudden death. The primary event in this syndrome is inhibition of the rapid component of the delayed rectifying potassium current (IKr) by these compounds. The compounds bind to the aperture-forming alpha sub-units of the channel protein carrying this current—sub-units that are encoded by the human ether-a-go-go-related gene (hERG). Since IKr plays a key role in repolarisation of the cardiac action potential, its inhibition slows repolarisation and this is manifested as a prolongation of the QT interval. Whilst QT interval prolongation is not a safety concern per se, it carries a risk of cardiovascular adverse effects and in a small percentage of people it can lead to TdP and degeneration into ventricular fibrillation.

Generally, compounds of the present invention have low activity against the hERG-encoded potassium channel. In this regard, low activity against hERG in vitro is indicative of low activity in vivo.

It is also desirable for drugs to possess good metabolic stability in order to enhance drug efficacy. Stability against human microsomal metabolism in vitro is indicative of stability towards metabolism in vivo.

Because of their physiological and pathophysiological significance, there is a need for new potent mGluR agonists and antagonists that display a high selectivity for mGluR subtypes, particularly the Group I receptor subtype, most particularly the mGluR5.

The object of the present invention is to provide compounds exhibiting an activity at metabotropic glutamate receptors (mGluRs), especially at the mGluR5 receptor. In particular, the compounds according to the present invention are predominantly peripherally acting, i.e. have a limited ability of passing the blood-brain barrier.

### DESCRIPTION OF THE INVENTION

The present invention relates to a compound of formula I:

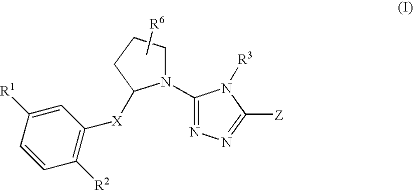

wherein
R¹ is methyl, halogen or cyano;
R² is hydrogen or fluoro;
R³ is C₁-C₃ alkyl or cyclopropyl;

### X is

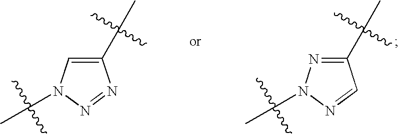

### Z is

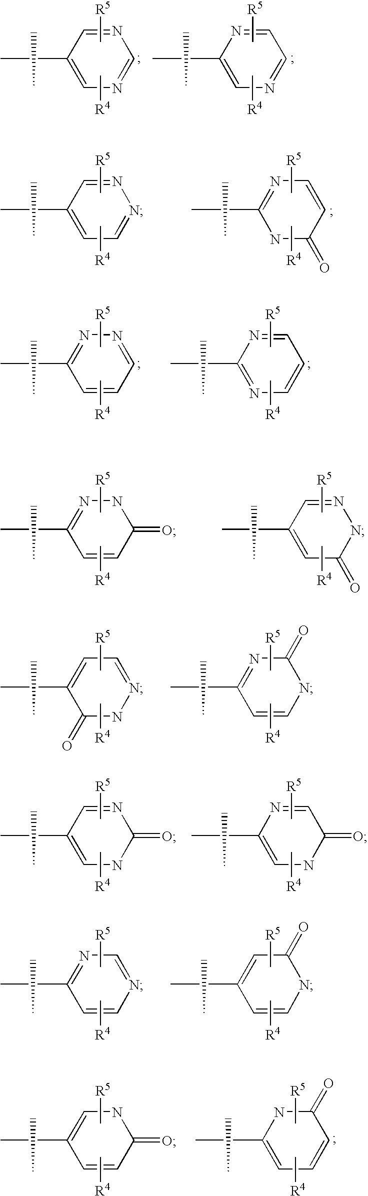

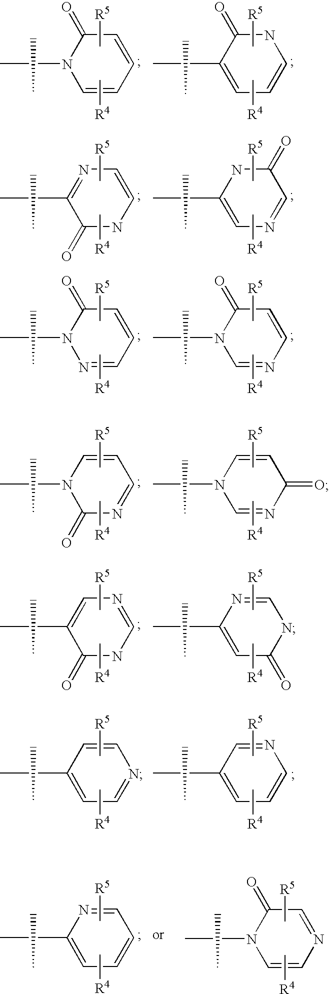

wherein
R⁴ is hydrogen, C₁-C₃ alkyl, fluoro or C₁-C₃ alkoxy;
R⁵ is hydrogen, C₁-C₃ alkyl, fluoro or C₁-C₃ alkoxy;
R⁶ is hydrogen, fluoro, C₁-C₃ alkyl, OR⁷ or NR⁷R⁸;
R⁷ is hydrogen or C₁-C₃ alkyl;
R⁸ is hydrogen or C₁-C₃ alkyl;
as well as pharmaceutically acceptable salts, hydrates, isoforms, tautomers and/or enantiomers thereof.

In one embodiment R¹ is halogen.

In a further embodiment, R¹ is chloro.

In a further embodiment, R² is hydrogen.

In a further embodiment, R³ is methyl.

In a further embodiment, R⁴ is methyl and R⁵ is hydrogen. In a further embodiment, R⁴ is hydrogen and R⁵ is hydrogen.

In a further embodiment, R⁶ is hydrogen or methyl. In a further embodiment, R⁶ is OR⁷, NR⁷R⁸.

In a further embodiment, R⁷ is hydrogen or methyl.

In a further embodiment, R⁸ is hydrogen or methyl.

In a further embodiment, Z is

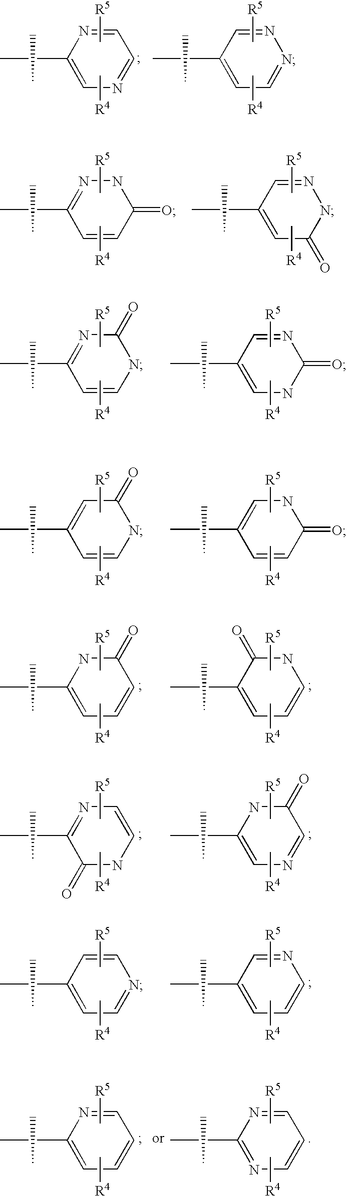

Another embodiment is a pharmaceutical composition comprising as active ingredient a therapeutically effective amount of the compound according to formula I, in association with one or more pharmaceutically acceptable diluents, excipients and/or inert carriers.

Other embodiments, as described in more detail below, relate to a compound according to formula I for use in therapy, in treatment of mGluR5 mediated disorders, in hie manufacture of a medicament for the treatment of mGluR5 mediated disorders.

Still other embodiments relate to a method of treatment of mGluR5 mediated disorders, comprising administering to a mammal a therapeutically effective amount of the compound according to formula I.

In another embodiment, there is provided a method for inhibiting activation of mGluR5 receptors, comprising treating a cell containing said receptor with an effective amount of the compound according to formula I.

The compounds of the present invention are useful in therapy, in particular for the treatment of neurological, psychiatric, pain, and gastrointestinal disorders.

It will also be understood by those of skill in the art that certain compounds of the present invention may exist in solvated, for example hydrated, as well as unsolvated forms. It will further be understood that the present invention encompasses all such solvated forms of the compounds of formula I.

Within the scope of the invention are also salts of the compounds of formula I. Generally, pharmaceutically acceptable salts of compounds of the present invention are obtained using standard procedures well known in the art, for example, by reacting a sufficiently basic compound, for example an alkyl amine with a suitable acid, for example, HCl, acetic acid or a methanesulfonic acid to afford a salt with a physiologically acceptable anion. It is also possible to make a corresponding alkali metal (such as sodium, potassium, or lithium) or an alkaline earth metal (such as a calcium) salt by treating a compound of the present invention having a suitably acidic proton, such as a carboxylic acid or a phenol, with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques. Additionally, quaternary ammonium salts can be prepared by the addition of alkylating agents, for example, to neutral amines.

In one embodiment of the present invention, the compound of formula I may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

The general terms used in the definition of formula I have the following meanings:

Halogen as used herein is selected from chlorine, fluorine, bromine or iodine.

C₁-C₃ alkyl is a straight or branched alkyl group, having from 1 to 3 carbon atoms, for example methyl, ethyl, n-propyl or isopropyl.

C₁-C₃ alkoxy is an alkoxy group having 1 to 3 carbon atoms, for example methoxy, ethoxy, isopropoxy or n-propoxy.

All chemical names were generated using ACDLABS 9.04.

In formula I above, X may be present in any of the two possible orientations.

### Pharmaceutical Composition

The compounds of the present invention may be formulated into conventional pharmaceutical compositions comprising a compound of formula I, or a pharmaceutically acceptable salt or solvate thereof, in association with a pharmaceutically acceptable carrier or excipient. The pharmaceutically acceptable carriers can be either solid or liquid. Solid form preparations include, but are not limited to, powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents. A solid carrier can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized moulds and allowed to cool and solidify.

Suitable carriers include, but are not limited to, magnesium carbonate, magnesium stearate, talc, lactose, sugar, pectin, dextrin, starch, tragacanth, methyl cellulose, sodium carboxymethyl cellulose, low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art. Exemplary compositions intended for oral use may contain one or more coloring, sweetening, flavoring and/or preservative agents.

Depending on the mode of administration, the pharmaceutical composition will include from about 0.05% w (percent by weight) to about 99% w, or from about 0.10/w to 50% w, of a compound of the invention, all percentages by weight being based on the total weight of the composition.

A therapeutically effective amount for the practice of the present invention can be determined by one of ordinary skill in the art using known criteria including the age, weight and response of the individual patient, and interpreted within the context of the disease which is being treated or which is being prevented.

### Medical Use

The compounds according to the present invention are useful in the treatment of conditions associated with excitatory activation of mGluR5 and for inhibiting neuronal damage caused by excitatory activation of mGluR5. The compounds may be used to produce an inhibitory effect of mGluR5 in mammals, including man.

The Group I mGluR receptors including in mGluR5 are highly expressed in the central and peripheral nervous system and in other tissues. Thus, it is expected that the compounds of the invention are well suited for the treatment of mGluR5-mediated disorders such as acute and chronic neurological and psychiatric disorders, gastrointestinal disorders, and chronic and acute pain disorders.

The invention relates to compounds of formula I, as defined herein before, for use in therapy.

The invention relates to compounds of formula I, as defined herein before, for use in treatment of mGluR5-mediated disorders.

The invention relates to compounds of formula I, as defined herein before, for use in treatment of Alzheimer's disease senile dementia, AIDS-induced dementia, Parkinson's disease, amylotropic lateral sclerosis, Huntington's Chorea, migraine, epilepsy, schizophrenia, depression, anxiety, acute anxiety, opthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of formula I, as defined above, for use in treatment of pain related to migraine, inflammatory pain, neuropathic pain disorders such as diabetic neuropathies, arthritis and rheumatoid diseases, low back pain, post-operative pain and pain associated with various conditions including cancer, angina, renal or billiary colic, menstruation, migraine and gout.

The invention relates to compounds of formula I as defined herein before, for use in treatment of stroke, head trauma, anoxic and ischemic injuries, hypoglycemia, cardiovascular diseases and epilepsy.

The present invention relates also to the use of a compound of formula I as defined herein before, in the manufacture of a medicament for the treatment of mGluR Group I receptor-mediated disorders and any disorder listed above.

One embodiment of the invention relates to the use of a compound according to formula I in the treatment of gastrointestinal disorders.

Another embodiment of the invention relates a compound of formula I for the inhibition of transient lower esophageal sphincter relaxations, for the treatment of GERD, for the prevention of gastroesophageal reflux, for the treatment regurgitation, for treatment of asthma, for treatment of laryngitis, for treatment of lung disease, for the management of failure to thrive, for the treatment of irritable bowel syndrome (IBS) and for the treatment of functional dyspepsia (FD).

Another embodiment of the invention relates to the use of a compound of formula I for the manufacture of a medicament for inhibition of transient lower esophageal sphincter relaxations, for the treatment of GERD, for the prevention of gastroesophageal reflux, for the treatment regurgitation, for treatment of asthma, for treatment of laryngitis, for treatment of lung disease, for the management of failure to thrive, for the treatment of irritable bowel syndrome (IBS) and for the treatment of functional dyspepsia (FD).

Another embodiment of the present invention relates to the use of a compound of formula I for treatment of overactive bladder or urinary incontinence.

The wording “TLESR”, transient lower esophageal sphincter relaxations, is herein defined in accordance with Mittal, R. K, Holloway, R. H, Penagini, R., Blackshaw, L. A., Dent, J., 1995; Transient lower esophageal sphincter relaxation. Gastroenterology 109, pp. 601-610.

The wording “reflux” is herein defined as fluid from the stomach being able to pass into the esophagus, since the mechanical barrier is temporarily lost at such times.

The wording “GERD”, gastro-esophageal reflux disease, is herein defined in accordance with van Heerwarden, M. A., Smout A. J. P. M, 2000; Diagnosis of reflux disease. Baillière's Clin. Gastroenterol. 14, pp. 759-774.

The compounds of formula I above are useful for the treatment or prevention of obesity or overweight, (e.g., promotion of weight loss and maintenance of weight loss), prevention or reversal of weight gain (e.g., rebound, medication-induced or subsequent to cessation of smoking), for modulation of appetite and/or satiety, eating disorders (e.g. binge eating, anorexia, bulimia and compulsive) and cravings (for drugs, tobacco, alcohol, any appetizing macronutrients or non-essential food items).

The invention also provides a method of treatment of mGluR5-mediated disorders and any disorder listed above, in a patient suffering from, or at risk of, said condition, which comprises administering to the patient an effective amount of a compound of formula I, as herein before defined.

The dose required for the therapeutic or preventive treatment of a particular disorder will necessarily be varied depending on the host treated, the route of administration and the severity of the illness being treated.

In the context of the present specification, the term “therapy” and “treatment” includes prevention or prophylaxis, unless there are specific indications to the contrary. The terms “therapeutic” and “therapeutically” should be construed accordingly.

In this specification, unless stated otherwise, the term “antagonist” and “inhibitor” shall mean a compound that by any means, partly or completely, blocks the transduction pathway leading to the production of a response by the ligand.

The term “disorder”, unless stated otherwise, means any condition and disease associated with metabotropic glutamate receptor activity.

One embodiment of the present invention is a combination of a compound of formula I and an acid secretion inhibiting agent. A “combination” according to the invention may be present as a “fix combination” or as a “kit of parts combination”. A “fix combination” is defined as a combination wherein the (i) at least one acid secretion inhibiting agent; and (ii) at least one compound of formula I are present in one unit. A “kit of parts combination” is defined as a combination wherein the (i) at least one acid secretion inhibiting agent; and (ii) at least one compound of formula I are present in more than one unit. The components of the “kit of parts combination” may be administered simultaneously, sequentially or separately. The molar ratio of the acid secretion inhibiting agent to the compound of formula I used according to the invention in within the range of from 1:100 to 100:1, such as from 1:50 to 50:1 or from 1:20 to 20:1 or from 1:10 to 10:1. The two drugs may be administered separately in the same ratio. Examples of acid secretion inhibiting agents are H2 blocking agents, such as cimetidine, ranitidine; as well as proton pump inhibitors such as pyridinylmethylsulfinyl benzimidazoles such as omeprazole, esomeprazole, lansoprazole, pantoprazole, rabeprazole or related substances such as leminoprazole.

### Non-Medical Use

In addition to their use in therapeutic medicine, the compounds of formula I, as well as salts and hydrates of such compounds, are useful as pharmacological tools in the development and standardisation of in vitro and in vivo test systems for the evaluation of the effects of inhibitors of mGluR related activity in laboratory animals such as cats, dogs, rabbits, monkeys, rats and mice, as part of the search for new therapeutic agents.

### Methods of Preparation

Another aspect of the present invention provides processes for preparing compounds of formula I, or salts or hydrates thereof. Processes for the preparation of the compounds in the present invention are described herein.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include for example, straight and reversed phase chromatography on column or rotating plate, recrystallisation, distillation and liquid-liquid or solid-liquid extraction, which will be readily understood by the one skilled in the art. The definitions of substituents and groups are as in formula I except where defined differently. The term “room temperature” and “ambient temperature” shall mean, unless otherwise specified, a temperature between 16° C. and 25° C.

The term “reflux” shall mean, unless otherwise stated, in reference to an employed solvent a temperature at or above the boiling point of named solvent.

### Abbreviations

aq. Aqueous

### DCM Dichloromethane

### DMF Dimethylformamide

### DMSO Dimethylsulfoxide

EtOAc Ethyl acetate

### EtOH Ethanol

h hour(s)
HPLC High performance liquid chromatography
i-PrOH Isopropanol
LCMS HPLC mass spec

### LG Leaving Group

### MeCN Acetonitrile

### MeOH Methanol

### min Minutes

### MeI Iodomethane

### Me Methyl

NMR Nuclear magnetic resonance
o.n. Over night
PG Protection group

### THF Tetrahydrofurane

TFA Trifluoroacetic acid

### Preparation of Intermediates

The intermediates provided in synthetic paths given below, are useful for further preparation of compounds of formula I. Other starting materials are either commercially available or can be prepared via methods described in the literature. The synthetic pathways described below are non-limiting examples of preparations that can be used. One of skill in the art would understand other pathways might be used.

### Synthesis of Alkynes

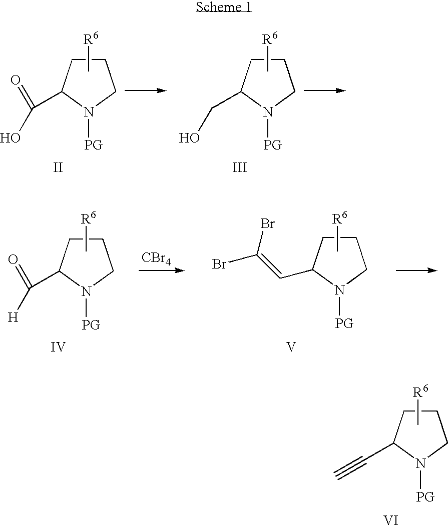

An aldehyde compound of formula IV, wherein R⁶ is defined in formula I, may be synthesized from a protected proline of formula I through the reduction of a carboxylic acid and the oxidation of the resulting methylene alcohol III by procedures well known to a person skilled in the art and described in the literature. Aldehydes of formula IV in an inert solvent such as DCM may be treated with triphenylphosphine and carbontetrabromide in an inert solvent such as DCM to give dibromo alkene of formula V, (J. Med. Chem, (1992), 35 (9), 1550-7), which in an ether solvent such as THF is reacted at 78° C., 0° C. with an alkyl lithium reagent such as sec-butyllithium to give the alkyne VI; (Eur. Pat. Appl., 408879, 23 Jan. 1991 for the alkyne synthesis).

### Synthesis of 1,2,3-triazoles

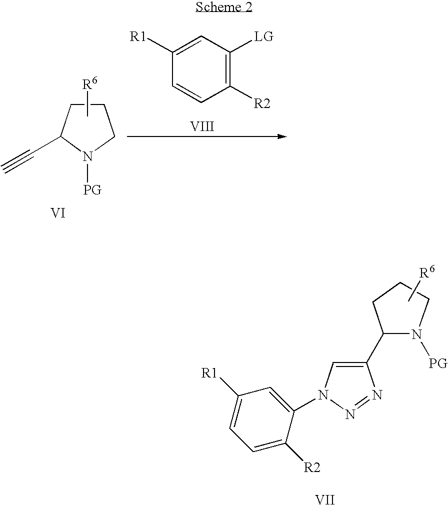

Alkyne VI may be transformed into VII e.g. by treatment of compound VI with a halogenated substituted phenyl of formula VIII (scheme 2 wherein LG=I) with sodium azide and a copper-catalysts (see J. Org. Chem., (2002), 67, 3057) in a solvents mixture like DMSO/H₂O at 20° C.-100° C.

An alternative regioisomer such as X, scheme 3, may be synthesized either from a substituted triazole IX which may undergo a nucleophilic addition with a halogenated phenyl such as VII (scheme 3, LG=F), using an inorganic base such as K₂CO₃ in DMSO (Tetrahedron, (2001), 57 (22), 4781-4785), or from an α-hydroxyketone XI which may be reacted with an aryl hydrazine in the precense of e.g. cupnic chloride and heating (Synthetic Commun., (2006), 36, 2461 2468).

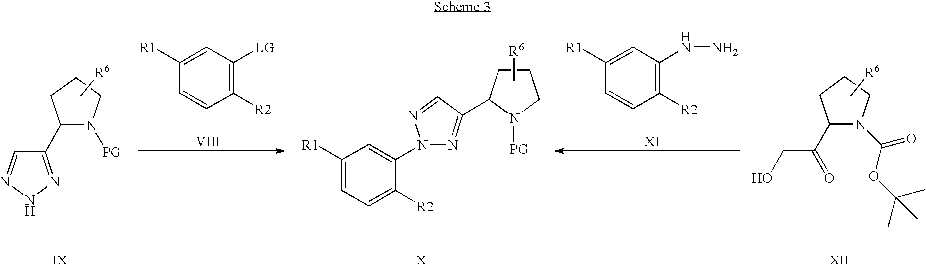

### Synthesis of Pyrrolidino Triazoles

The amine of formula XIV, which may be obtained from a suitably protected compound of formula XIII (scheme 4, e.g. wherein PG is Boc) by deprotection (e.g. TFA in DCM at r.t.), may be subjected to a sequence of thiourea formation to give a compound of formula XV, methylation XVI and triazole formation to deliver compounds of formula I. Thioureas of formula XV are available from well established methods using for example an isothiocyanate MeNCS, in a solvent such as MeOH, EtOH and the like, at a temperature between room temperature and 100° C. Alkylation of the thiourea intermediates can be performed using an alkylating agent such as Met in a solvent such as DMF, acetone, to DCM, THF at room temperature or elevated temperatures to give the isothiourea of formula XIII. A compounds of formula XIII may react with acyl hydrazine to form an intermediate which may be cyclized to the triazoles of formula I by heating at 0° C. to 150° C. in a suitable solvent such as pyridine, IPA or DMSO.

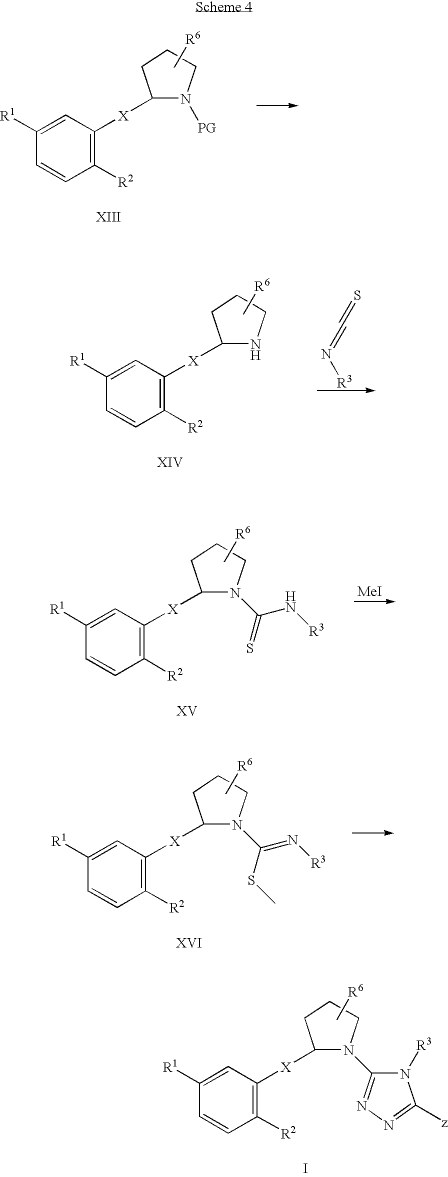

### EXAMPLES

The invention will now be illustrated by the following non-limiting examples.

### General Methods

All starting materials are commercially available or earlier described in the literature. The ¹H and ¹³C NMR spectra were recorded either on Varian Mercery Plus or Varian INOVA spectrometers operating at 300, 400 and 600 MHz for ¹H NMR respectively, using TMS or the residual solvent signal as reference, in deuterated chloroform as solvent unless otherwise indicated. All reported chemical shifts are in ppm on the delta-scale, and the fine splitting of the signals as appearing in the recordings (s: singlet, br s: broad singlet, d: doublet, t: triplet, q: quartet, m: multiplet).

Analytical in line liquid chromatography separations followed by mass spectra detections, were recorded on a Waters LCMS consisting of an Alliance 2795 (LC) and a ZQ single quadropole mass spectrometer. The mass spectrometer was equipped with an electrospray ion source operated in a positive and/or negative ion mode. The ion spray voltage was ±3 kV and the mass spectrometer was scanned from m/z 100-700 at a scan time of 0.8 s. To the column, SunFire C18 2.5μ 3×20 mm was applied a linear gradient from 5% to 100% MeCN in a pH 3: formiate buffer or a pH 7: acetate buffer.

Preparative reversed phase chromatography was run on a Waters Delta Prep Systems with a diode array detector using an Kromasil C8, 10 μm columns. Purification of products were also done by flash chromatography in silica-filled glass columns. Microwave heating was performed in a Smith Synthesizer Single-mode microwave cavity producing continuous irradiation at 2450 MHz (Personal Chemistry AB, Uppsala, Sweden).

### General Procedure for Synthesis of Acylhydrazines

The acylhydrazines (XI) which are applied in scheme 3 are commercially available or can be synthesised from the corresponding alkyl esters by heating with hydrazine in a solvent such as MeOH, EtOH or THF at a temperature from 50° C. to 100° C.

### Example 1

### Tert-Butyl (2R)-2-(2,2-dibromovinyl)pyrrolidine-1-carboxylate

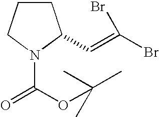

The title compound was synthesized according to the literature, here reported with new NMR data in CDCl₃. (J. Med. Chem., (1992), 35 (9), 1550-7).

¹H NMR (500 MHz, CDCl₃): δ 6.34 (m, 1H), 4.35 (m, 1H), 3.32-3.49 (m, 2H), 2.14 (m, 1H), 1.83 (m, 2H), 1.71 (m, 1H), 1.52 (s, 2H)*, 1.44 (m, 7H)*.

*possible rotamers

### Example 2

### Tert-Butyl (2R)-2-ethynylpyrrolidine-1-carboxylate

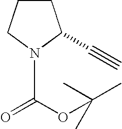

The title compound was synthesized according to the literature (Eur. Pat. Appl., 408879, 23 Jan. 1991).

¹H NMR (400 MHz, CDCl₃): δ 4.37-4.47 (m, 1H)*, 3.27-3.41 (m, 2H), 1.85-2.17 (m, 5H), 1.43 (s, 9H).

*possible rotamers

### Example 3

### Tert-Butyl (2R)-2-[1-(3-chlorophenyl)-1H-1,2,3-triazol-4-yl]pyrrolidine-1-carboxylate

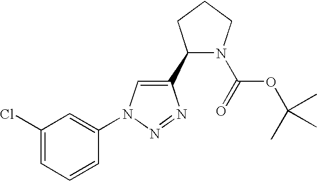

A mixture of the title compound from Example 2 (6.47 g, 33.1 mmol), 3-chloro-iodobenzene (7.90 g, 33.1 mmol), NaN₃ (2.85 mg, 43.8 mmol), CuSO₄.5H₂O (0.49 g, 2.0 mmol), sodium ascorbate (0.92 g, 4.6 mmol), L-proline (0.78 mg, 6.8 mmol), Na₂CO₃ (0.88 g, 8.3 mmol), DMSO (180 mL) and 1120 (20 mL) was stirred o.n. at 65° C. The resulting mixture was poured into 10% aqueous NH₄OH solution and extracted with ethyl acetate (3×200 mL). The organic layers were combined, washed with brine, dried over anhydrous Na₂SO₄ and subjected to silica gel column to afford the title compound (9.50 g, 82%).

¹H NMR (400 MHz, CDCl₃): δ 7.90 (s, broad, 1H), 7.74 (m, 1H), 7.59 (m, 1H), 7.32-7.48 (m, 2H), 5.07 (m, 1H), 2.06-2.33 (m, 2H), 1.85-2.52 (m, 4H), 1.43 (s, 5H)*, 1.35 (m, 4H)*.

*possible rotamers

### Example 4

### 1-(3-Chlorophenyl)-4-[(2R)-pyrrolidin-2-yl]-1H-1,2,3-triazole

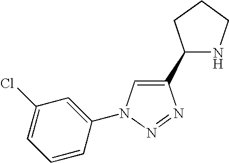

TFA (27 mL) was added to the title compound of Example 3 (9.49 g, 27.2 mmol) in DCM (54 mL) at room temperature. The resulting mixture was stirred for 2 h. DCM and excess TFA were removed in vacuo. DCM was added and the solution was washed with aq. 1M NaOH, concentrated and dried under vacuum pump to give amine (6.84 g, 100%).

¹H NMR (400 MHz, CDCl₃): δ 7.98 (s, 1H), 7.75 (m, 1H), 7.60 (m, 1H), 7.34-7.44 (m, 2H), 4.46 (t, 1H), 3.17 (m, 1H)*, 3.07 (m, 1H)*, 2.90 (s, broad, 1H), 2.22-2.36 (m, 1H), 1.85-2.05 (m, 3H).

*possible rotamers

### Example 5

### (2R)-2-[1-(3-Chlorophenyl)-1H-1,2,3-triazol-4-yl]-N-methylpyrrolidine-1-carbothioamide

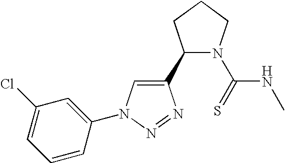

Methylisothiocyanate (3.12 g, 42.6 mmol) was added to an ice-cooled solution of the title compound of Example 4 (6.84 g, 27.5 mmol) in DCM (40 mL). Then the resulting mixture was stirred at room temperature o.n. DCM was removed in vacuo. The crude product was subjected to silica gel column to afford the title compound (7.32 g, 83%).

¹H NMR (400 MHz, CDCl₃) δ 8.07 (s, 1H), 7.74 (m, 1H), 7.61 (m, 0.5H)*, 7.59 (m, 0.5H)*, 7.36-7.45 (m, 2H), 5.88 (s, broad, 1H), 5.61 (s, broad, 1H), 3.71 (m, 2H), 3.09 (s, 3H), 2.08-2.50 (m, 4H).

*possible rotamers

### Example 6

### Methyl (2R)-2-[1-(3-chlorophenyl)-1H-1,2,3-triazol-4-yl]-N-methylpyrrolidine-1-carbimidothioate

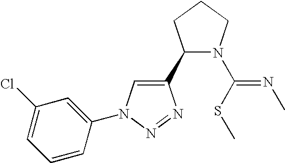

To the title compound of Example 5 (3.99 g, 12.4 mmol) in THF (30 mL) at room temperature were added sodium tert-butoxide (1.32 g, 13.7 mmol) and MeI (2.47 g, 17.4 mmol). The reaction mixture was stirred for 1.5 h. Then to the mixture were added sodium tert-butoxide (0.11 g, 0.34 mmol), MeI (0.38 g, 2.68 mmol) and THF (10 mL). The resulting mixture was stiffed another 2 h. THF and excess MeI were removed in vacuo. The resulting residue was mixed with water and then extracted with DCM. The organic layer was washed with water and brine, dried over anhydrous Na₂SO₄, filtered and concentrated to give the title product as a pink solid (4.15 g, 90%).

¹H NMR (400 MHz, CDCl₃): δ 7.72-7.76 (m, 2H), 7.61 (m, 0.5H)*, 7.59 (m, 0.5H)*, 7.34-7.45 (m, 2H), 5.41 (s, broad, 1H), 3.69 (m, 1H), 3.61 (m, 1H), 3.21 (s, 3H), 2.32 (m, 2H), 2.25 (s, 3H), 1.98 (m, 2H).

*possible rotamers

### Example 7

### 6-Oxo-1,6-dihydropyridazine-4-carbohydrazide

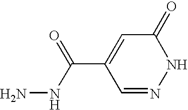

The subtitle compound of step 7C was heated with hydrazine hydrate (1.2 eq.) at 78° C. o.n. The reaction mixture was cooled and concentrated in vacuo. The residue was triturated with EtOAc, filtered and dried to give the title product (99%).

¹H-NMR (400 MHz, (CD₃)₂SO): δ 8.05 (d, 1H), 7.09 (d, 1H), 6.40 (broad s, 4H).

### Step 7A: 5-Methylpyridazin-3(2H)-one

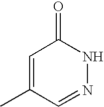

The 4,4-dimethoxy-3-methyl-but-2-enoic acid ethyl ester (Qi-Ying Hu, Pankaj D. Rege, and E. J. Corey, J. Am. Chem. Soc., (2004), 126, 5984) (82 g, 440 mmol) was mixed with hydrazine hydrate (50 g, 999 mmol) at room temperature. The mixture was heated at 60° C. for 4 h. After evaporation of solvents the oil residue was further dried under vacuum. To the resulting residue was added 6 M aq. HCl. The mixture was heated at 60° C. for 5 h. The solvents were removed in vacuo. To the residue was added MeOH three times, followed by concentration in vacuo. To the resulting residue was treated with dry ethanol followed by filtration to removed insoluble solid. The filtrate was concentrated to dryness. To the resulting residue was added dry IPA and 20 g anhydrous K₂CO₃. The mixture was heated to for 20 min at 60° C. After filtration, the filtrate was concentrated to dryness. The residue was purified with flash chromatography using DCM:MeOH:Et₃N (10:1:0.3) to give the subtitle compound (13.4 g, 28%).

¹H NMR (400 MHz, CD₃OD): δ 2.24 (s, 3H), 6.73 (s, 1H), 7.82 (s, 1H).

### Step 7B: 6-Oxo-1,6-dihydropyridazine-4-carboxylic Acid

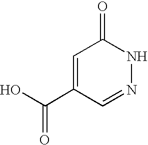

To a stirred solution of the subtitle compound of Step 7A (4.4 g, 40 mmol) in concentrated sulphuric acid (80 mL), potassium dichromate (18 g, 61 mmol) was added in small quantities at 50-60° C. as a finely ground powder. The starting material was added to the mixture within 20 min. Stirring was continued for a further 10 min at 60° C., then the viscous green mixture was poured on crushed ice. The solid powder, which separated, was collected, washed with cold water and dried to give the subtitle compound (4.5 g, 77%).

¹H NMR (400 MHz, (CD₃)₂SO): δ 7.22 (s, 3H), 8.13 (s, 1H), 13.38 (s, broad, 1H).

### Step 7C: Ethyl 6-oxo-1,6-dihydropyridazine-4-carboxylate

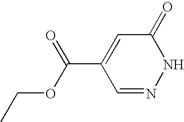

The subtitle product of Step 7B was dissolved in EtOH (10 mL) and concentrated H₂SO₄ (4.2 mL) was added and then heated at reflux for 5 hours. The reaction mixture was cooled, concentrated in vacuo and basified with saturated Na₂CO₃. After filtration, the aqueous phase was extracted with EtOAc, dried over anhydrous Na₂SO₄, filtered and concentrated to give the subtitle compound (83%).

¹H NMR (400 MHz, CD₃OD): δ 8.27 (d, 1H), 7.42 (d, 1H), 4.40 (q, 2H), 1.39 (t, 3H).

### Example 8.1

### 4-(5-{(2R)-2-[1-(3-Chlorophenyl)-1H-1,2,3-triazol-4-yl]pyrrolidin-1-yl}-4-methyl-4H-1,2,4-triazol-3-yl)pyridine

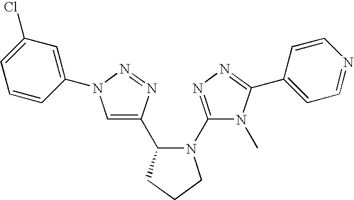

A mixture of the title compound of Example 7 (2.85 g, 8.49 mmol) and isonicotinic acid to hydrazide (1.37 g, 9.99 mmol) in DMSO (16 mL) was heated at 120° C. for 2 h. The crude product was purified by automatic preparative HPLC using a gradient of acetonitrile/5% acetonitrile-water phase containing 0.2% NH₄OH to give the title compound (2.99 g, 87%).

¹H NMR (400 MHz, CDCl₃): δ 8.66 (m, 2H), 8.05 (s, 1H), 7.71 (m, 1H), 7.48-7.57 (m, 3H), 7.29-7.42 (m, 2H), 5.40 (t, 1H), 3.92 (m, 1H), 3.54 (s, 3H), 3.44 (m, 1H), 2.58 (m, 1H), 2.45 (m, 1H), 2.05-2.31 (m, 2H).

In a similar manner the following compounds were synthesized:

| Example | Structure | Name | Yield |
|---|---|---|---|
| 8.2 | 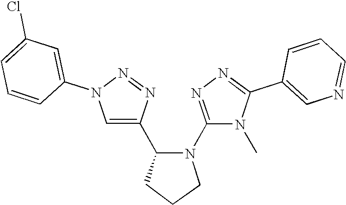 | 3-(5-{(2R)-2-[1-(3-chlorophenyl)-1H-1,2,3-triazol-4-yl]pyrrolidin-1-yl}-4-methyl-4H-1,2,4-triazol-3-yl)pyridine | 61%57 mg |
| ¹H NMR | (600 MHz, (CD₃)₂SO): δ 8.82 (m, 1 H), 8.74 (m, 1 H), 8.63 (m, 1 H), 8.03 (m, | | |
| | 1 H), 7.99 (m, 1 H), 7.87 (m, 1 H), 7.57 (t, 1 H), 7.51 (m, 2 H), 5.33 (m, 1 H), | | |
| | 3.78 (m, 1 H), 3.54 (s, 3 H), 3.43 (m, 1 H), 2.40 (m, n.d)*, 1.98-2.14 (m, 2 H) | | |
| 8.3 | 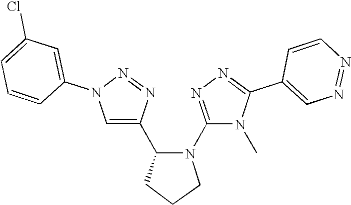 | 4-(5-{(2R)-2-[1-(3-chlorophenyl)-1H-1,2,3-triazol-4-yl]pyrrolidin-1-yl}-4-methyl-4H-1,2,4-triazol-3-yl)pyridine | 17%76 mg |
| ¹H NMR | (500 MHz, CD₃OD): δ 9.52 (m, 1 H), 9.30 (m, 1 H), 8.48 (s, 1 H), 7.97 (m, | | |
| | 1 H), 7.89 (m, 1 H), 7.76 (m, 1 H), 7.52 (t, 1 H), 7.46 (m, 1 H), 5.38 (m, 1 H), | | |
| | 3.98 (m, 1 H), 3.72 (s, 3 H), 3.56 (m, 1 H), 2.54 (m, 1 H), 2.10-3.34 (m, 3 H) | | |
| 8.4 | 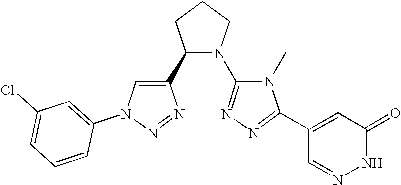 | 5-(5-{(2R)-2-[1-(3-chlorophenyl)-1H-1,2,3-triazol-4-yl]pyrrolidin-1-yl}-4-methyl-4H-1,2,4-triazol-3-yl)pyridin-3(2H)-one | 41%41 mg |

| | |
|---|---|
| ¹H NMR | (500 MHz, CDCl₃): δ 8.49 (s, 1 H), 8.28 (m, 1 H), 7.91 (m, 1 H), 7.78 (m, |
| | 1 H), 7.54 (t, 1 H), 7.48 (m, 1 H), 7.16 (m, 1 H), 5.39 (m, 1 H), 3.98 (m, 1 H), |
| | 3.71 (s, 3 H), 3.56 (m, 1 H), 2.55 (m, 1 H), 2.10-2.35 (m, 3 H) |

*overlaps with residual solvent peak.

### Biological Evaluation

Functional Assessment of mGluR5 Antagonism in Cell Lines Expressing mGluR5D

The properties of the compounds of the invention can be analyzed using standard assays for pharmacological activity. Examples of glutamate receptor assays are well known in the art as described in for example Aramori et al, Neuron 8:757 (1992), Tanabe et al., Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay (FLIPR) that measures the mobilization of intracellular calcium, [Ca²⁺]ᵢ in cells expressing mGluR5 or another assay (IP3) that measures inositol phosphate turnover.

### FLIPR Assay

Cells expressing human mGluR5d as described in WO97/05252 cultured in a mixture of high glucose DMEM with Glutamax (31966-021) (500 mL), 10% dialyzed fetal bovine serum (Hyclone #SH30079.03) (56 mL), 200 μg/mL Hygromycin B (Invitrogen 45-0430, 50 mg/mL) (2.2 mL), 200 μg/mL Zeocin (Invitrogen #R250-01; 100 mg/mL) (1.1 mL) are seeded at a density of 100,000 cells per well on collagen coated clear bottom 96-well plates with black sides and cells were allowed to adhere over night before experiments. All assays are done in a buffer containing 146 mM NaCl, 5 mM KCl, 1 mM MgCl₂, 1 mM CaCl₂, 20 mM HEPES, 1 mg/mL glucose and 1 mg/mL BSA Fraction IV (pH 7.4). Cell cultures in the 96-well plates are loaded for 60 minutes in the above mentioned buffer containing 6 μM of the acetoxymethyl ester form of the fluorescent calcium indicator fluo-3 (Molecular Probes, Eugene, Oreg.) in 0.025% pluronic acid (a proprietary, non-ionic surfactant polyol—CAS Number 9003-11-6). Following the loading period the fluo-3 buffer is removed and replaced with fresh assay buffer. FLIPR experiments are done using a laser setting of 0.700 W and a 0.4 second CCD camera shutter speed with excitation and emission wavelengths of 488 nm and 562 nm, respectively. Each experiment is initiated with 160 μl of buffer present in each well of the cell plate. A 40 μl addition from the antagonist plate was followed by a 50 μL addition from the agonist plate. A 30 minutes, in dark at 25° C., interval separates the antagonist and agonist additions. The fluorescence signal is sampled 50 times at 1-second intervals followed by 3 samples at 5-second intervals immediately after each of the two additions. Responses are measured as the difference between the peak heights of the response to agonist, less the background fluorescence within the sample period. IC₅₀ determinations are made using a linear least squares fitting program.

### IP3 Assay

An additional functional assay for mGluR5d is described in WO97/05252 and is based on phosphatidylinositol turnover. Receptor activation stimulates phospholipase C activity and leads to increased formation of inositol 1,4,5,triphosphate (IP₃). GHEK stably expressing the human mGluR5d are seeded onto 24 well poly-L-lysine coated plates at 40×10⁴ cells/well in media containing 1 μCi/well [3H] myo-inositol. Cells were incubated overnight (16 h), then washed three times and incubated for 1 h at 37° C. in HEPES buffered saline (146 mM NaCl, 4.2 mM KCl, 0.5 mM MgCl₂, 0.1% glucose, 20 mM HEPES, pH 7.4) supplemented with 1 unit/mL glutamate pyruvate transaminase and 2 mM pyruvate. Cells are washed once in HEPES buffered saline and pre-incubated for 10 loin in HEPES buffered saline containing 10 mM LiCl. Compounds are incubated in duplicate at 37° C. for 15 min, then either glutamate (80 μM) or DHPG (30 μM) is added and incubated for an additional 30 min. The reaction is terminated by the addition of 0.5 mL perchloric acid (5%) on ice, with incubation at 4° C. for at least 30 min. Samples are collected in 15 mL polypropylene tubes and inositol phosphates are separated using ion-exchange resin (Dowex AG1-X8 formate form, 200-400 mesh, BIORAD) columns. Inositol phosphate separation was done by first eluting glycero phosphatidyl inositol with 8 mL 30 mM ammonium formate. Next, total inositol phosphates is eluted with 8 mL 700 mM ammonium formate/100 mM formic acid and collected in scintillation vials. This eluate is then mixed with 8 mL of scintillant and [3H] inositol incorporation is determined by scintillation counting. The dpm counts from the duplicate samples are plotted and IC₅₀ determinations are generated using a linear least squares fitting program.

### Abbreviations

### BSA Bovine Serum Albumin

### CCD Charge Coupled Device

### CRC Concentration Response Curve

### DHPG 3,5-Dihydroxyphenylglycine

### DPM Disintegrations per Minute

### EDTA Ethylene Diamine Tetraacetic Acid

FLIPR Fluorometric Imaging Plate reader

### GHEK GLAST-containing Human Embrionic Kidney

GLAST Glutamate/aspartate transporter
HEPES 4-(2-Hydroxyethyl)-1-piperazineethanesulfonic acid (buffer)
IP₃ Inositol triphosphate

Generally, the compounds were active in the assay above with IC₅₀ values less than 10 000 nM. In one aspect of the invention, the IC₅₀ value is less than 1 000 nM. In a further aspect of the invention, the IC₅₀ value is less than 100 nM.

### Determination of Brain to Plasma Ratio in Rat

Brain to plasma ratios are estimated in female Sprague Dawley rats. The compound is dissolved in water or another appropriate vehicle. For determination of brain to plasma ratio the compound is administrated as a subcutaneous, or an intravenous bolus injection, or an intravenous infusion, or an oral administration. At a predetermined time point after the administration a blood sample is taken with cardiac puncture. The rat is terminated by cutting the heart open, and the brain is immediately retained. The blood samples are collected in heparinized tubes and centrifuged within 30 minutes, in order to separate the plasma from the blood cells. The plasma is transferred to 96-well plates and stored at −20° C. until analysis. The brains are divided in halt and each half is placed in a pre-tarred tube and stored at −20° C. until analysis. Prior to the analysis, the brain samples are thawed and 3 mL/g brain tissue of distilled water is added to the tubes. The brain samples are sonicated in an ice bath until the samples are homogenized. Both brain and plasma samples are precipitated with acetonitrile. After centrifugation, the supernatant is diluted with 0.2% formic acid. Analysis is performed on a short reversed-phase HPLC column with rapid gradient elution and MSMS detection using a triple quadrupole instrument with electrospray ionisation and Selected Reaction Monitoring (SRM) acquisition. Liquid-liquid extraction may be used as an alternative sample clean-up. The samples are extracted, by shaking, to an organic solvent after addition of a suitable buffer. An aliquot of the organic layer is transferred to a new vial and evaporated to dryness under a stream of nitrogen. After reconstitution of the residuals the samples are ready for injection onto the HPLC column.

Generally, the compounds according to the present invention are peripherally restricted with a drug in brain over drug in plasma ratio in the rat of <0.5. In one embodiment, the ratio is less than 0.15.

### Determination of In Vitro Stability

Rat liver microsomes are prepared from Sprague-Dawley rats liver samples. Human liver microsomes are either prepared from human liver samples or acquired from BD Gentest. The compounds are incubated at 37° C. at a total microsome protein concentration of 0.5 mg/mL in a 0.1 mol/L potassium phosphate buffer at pH 7.4, in the presence of the cofactor, NADPH (1.0 mmol/L). The initial concentration of compound is 1.0 μmol/L. Samples are taken for analysis at 5 time points, 0, 7, 15, 20 and 30 minutes after the start of the incubation. The enzymatic activity in the collected sample is immediately stopped by adding a 3.5 times volume of acetonitrile. The concentration of compound remaining in each of the collected samples is determined by means of LC-MS. The elimination rate constant (k) of the mGluR5 inhibitor is calculated as the slope of the plot of In[mGluR5 inhibitor] against incubation time (minutes). The elimination rate constant is then used to calculate the half-life (T ½) of the mGluR5 inhibitor, which is subsequently used to calculate the intrinsic clearance (CLint) of the mGluR5 inhibitor in liver microsomes as: CLint.=(ln 2×incubation volume)/(T ½×protein concentration)=μl/min/mg

### Screening for Compounds Active Against TLESR

Adult Labrador retrievers of both genders, trained to stand in a Pavlov sling, are used. Mucosa-to-skin esophagostomies are formed and the dogs are allowed to recover completely before any experiments are done.

### Motility Measurement

In brief, after fasting for approximately 17 h with free supply of water, a multilumen sleeve/sidehole assembly (Dentsleeve, Adelaide, South Australia) is introduced through the esophagostomy to measure gastric, lower esophageal sphincter (LES) and esophageal pressures. The assembly is perfused with water using a low-compliance manometric perfusion pump (Dentsleeve, Adelaide, South Australia). An air-perfused tube is passed in the oral direction to measure swallows, and an antimony electrode monitored pH, 3 cm above the LES. All signals are amplified and acquired on a personal computer at 10 Hz.

When a baseline measurement free from fasting gastric/LES phase III motor activity has been obtained, placebo (0.9% NaCl) or test compound is administered intravenously (i.v., 0.5 mL/kg) in a foreleg vein. Ten min after i.v. administration, a nutrient meal (10% peptone, 5% D-glucose, 5% Intralipid, pH 3.0) is infused into the stomach through the central lumen of the assembly at 100 mL/min to a final volume of 30 mL/kg. The infusion of the nutrient meal is followed by air infusion at a rate of 500 mL/min until an intragastric pressure of 10±1 mmHg is obtained. The pressure is then maintained at this level throughout the experiment using the infusion pump for further air infusion or for venting air from the stomach. The experimental time from start of nutrient infusion to end of air insufflation is 45 min. The procedure has been validated as a reliable means of triggering TLESRs.

TLESRs is defined as a decrease in lower esophageal sphincter pressure (with reference to intragastic pressure) at a rate of >1 mmHg/s. The relaxation should not be preceded by a pharyngeal signal≦2 s before its onset in which case the relaxation is classified as swallow-induced. The pressure difference between the LES and the stomach should be less than 2 mmHg, and the duration of the complete relaxation longer than 1 s.

### Specimen Results are Shown in the Following Table:

| | | Brain/Plasma Ratio |
|---|---|---|
| Example | FLIPR hmGluR5d (nM) | of compound in Rat |
| 8.1 | 58 | 0.03 |
| 8.2 | 116 | <0.015 |
| 8.3 | 116 | 0.045 |
| 8.4 | 93 | <0.01 |

1. A compound of formula (I) 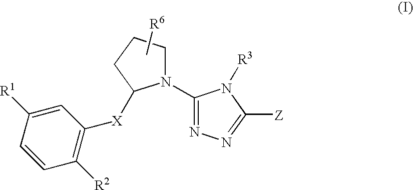
wherein
R¹ is methyl, halogen or cyano;
R² is hydrogen or fluoro;
R³ is C₁-C₃ alkyl or cyclopropyl;
X is 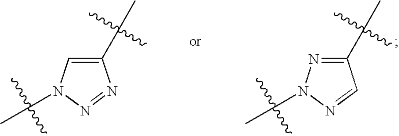
Z is 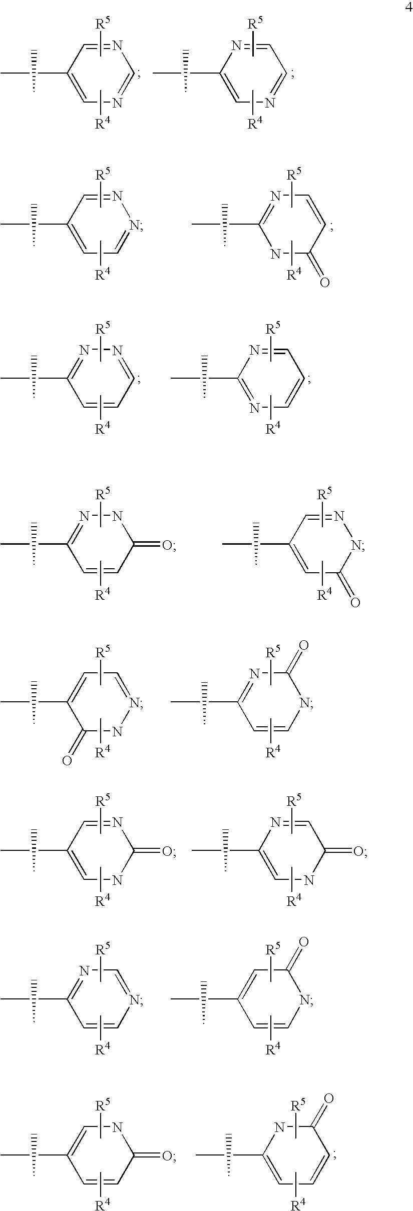 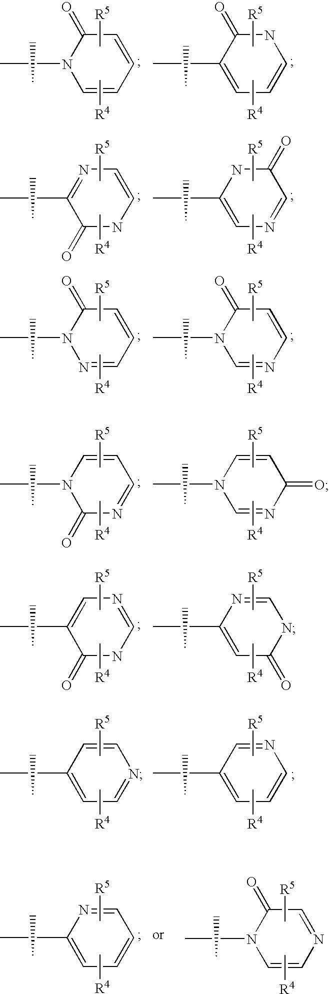
wherein
R⁴ is hydrogen, C₁-C₃ alkyl, fluoro or C₁-C₃ alkoxy;
R⁵ is hydrogen, C₁-C₃ alkyl, fluoro or C₁-C₃ alkoxy;
R⁶ is hydrogen, fluoro, C₁-C₃ alkyl, OR⁷ or NR⁷R⁸;
R⁷ is hydrogen or C₁-C₃ alkyl;
R⁸ is hydrogen or C₁-C₃ alkyl;
as well as pharmaceutically acceptable salts, hydrates, isoforms, tautomers and/or enantiomers thereof.

2. A compound according to claim 1, wherein R¹ is halogen.

3. A compound according to claim 2, wherein R¹ is chloro.

4. A compound according to any one of claims 1-3, wherein R² is hydrogen.

5. A compound according to claim 1, wherein R³ is methyl.

6. A compound according to claim 1, wherein R⁴ is methyl and R⁵ is hydrogen.

7. A compound according to claim 1, wherein R⁴ is hydrogen and R⁵ is hydrogen.

8. A compound according to claim 1, wherein R⁶ is hydrogen or methyl.

9. A compound according to claim 1, wherein R⁶ is OR⁷, NR⁷R⁸.

10. A compound according to claim 9, wherein R⁷ is hydrogen or methyl.

11. A compound according to claim 10, wherein R⁸ is hydrogen or methyl.

12. A compound according to claim 1, wherein Z is

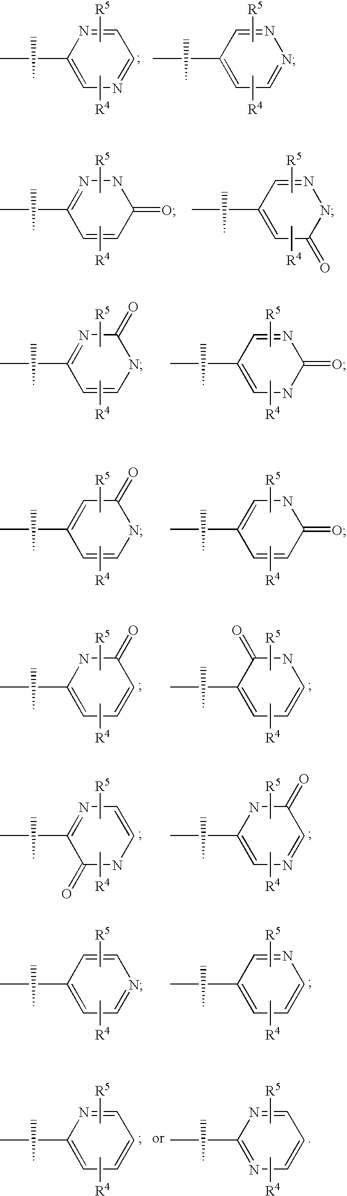

13. A compound according to claim 1, wherein
R¹ is halogen;
R² is hydrogen;
R³ is methyl;
R⁴ is hydrogen or methyl;
R⁵ is hydrogen or methyl;
R⁶ is hydrogen, fluoro, methyl, OR⁷ or NR⁸R⁸;
R⁷ is hydrogen or methyl;
R⁸ is hydrogen or methyl;
Z is 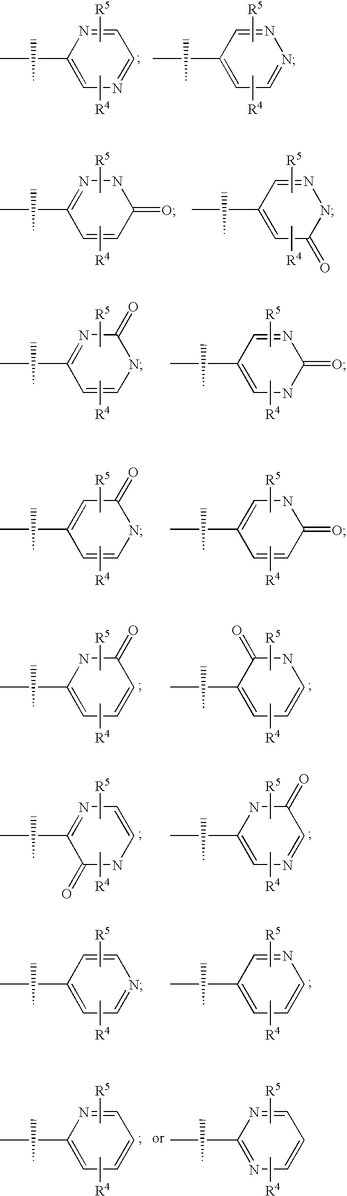
as well as pharmaceutically acceptable salts, hydrates, isoforms, tautomers and/or enantiomers thereof.

14. A compound according to claim 1 selected from
4-(5-{(2R)-2-[1-(3-Chlorophenyl)-1H-1,2,3-triazol-4-yl]pyrrolidin-1-yl}-4-methyl-4H-1,2,4-triazol-3-yl)pyridine;
3-(5-{(2R)-2-[1-(3-Chlorophenyl)-1H-1,2,3-triazol-4-yl]pyrrolidin-1-yl}-4-methyl-4H-1,2,4-triazol-3-yl)pyridine;
4-(5-{(2R)-2-[1-(3-Chlorophenyl)-1H-1,2,3-triazol-4-yl]pyrrolidin-1-yl}-4-methyl-4H-1,2,4 triazol-3-yl)pyridazine; and
5-(5-{(2R)-2-[1-(3-Chlorophenyl)-1H-1,2,3-triazol-4-yl]pyrrolidin-1-yl}-4-methyl-4H-1,2,4-triazol-3-yl)pyridazin-3(2H)-one;
as well as pharmaceutically acceptable salts, hydrates, isoforms, tautomers and/or enantiomers thereof.

15. A compound according to claim 1 for use in therapy.

16. A pharmaceutical composition comprising a compound according to claim 1 as an active ingredient, together with a pharmacologically and pharmaceutically acceptable carrier.

17. Use of a compound according to claim 1, or a pharmaceutically acceptable salt or an optical isomer thereof, for the manufacture of a medicament for the inhibition of transient lower esophageal sphincter relaxations.

18. Use of a compound according to claim 1, or a pharmaceutically acceptable salt or an optical isomer thereof, for the manufacture of a medicament for treatment or prevention of gastroesophageal reflux disease.

19. Use of a compound according to claim 1, or a pharmaceutically acceptable salt or an optical isomer thereof, for the manufacture of a medicament for treatment or prevention of pain.

20. Use of a compound according to claim 1, or a pharmaceutically acceptable salt or an optical isomer thereof, for the manufacture of a medicament for treatment or prevention of anxiety.

21. Use of a compound according to claim 1, or a pharmaceutically acceptable salt or an optical isomer thereof, for the manufacture of a medicament for treatment or prevention of irritable bowel syndrome (IBS).

22. A method for the inhibition of transient lower esophageal sphincter relaxations wherein an effective amount of a compound according to claim 1 is administered to a subject in need of such inhibition.

23. A method for the treatment or prevention of gastroesophageal reflux disease, wherein an effective amount of a compound according to claim 1 is administered to a subject in need of such treatment or prevention.

24. A method for the treatment or prevention of pain, wherein an effective amount of a compound according to claim 1 is administered to a subject in need of such treatment or prevention.

25. A method for the treatment or prevention of anxiety, wherein an effective amount of a compound according to claim 1 is administered to a subject in need of such treatment or prevention.

26. A method for the treatment or prevention of irritable bowel syndrome (IBS), wherein an effective amount of a compound according to claim 1 is administered to a subject in need of such treatment or prevention.

27. A combination comprising (i) at least one compound according to claim 1 and (ii) at least one acid secretion inhibiting agent.

28. A combination according to claim 27 wherein the acid secretion inhibiting agent is selected from cimetidine, ranitidine, omeprazole, esomeprazole, lansoprazole, pantoprazole, rabeprazole or leminoprazole.

29. A compound selected from
tert-Butyl (2R)-2-[1-(3-chlorophenyl)-1H-1,2,3-triazol-4-yl]pyrrolidine-1-carboxylate;
1-(3-Chlorophenyl)-4-[(2R)-pyrrolidin-2-yl]-1H-1,2,3-triazole;
(2R)-2-[1-(3-Chlorophenyl)-1H-1,2,3-triazol-4-yl]-N-methylpyrrolidine-1-carbothioamide; and
Methyl (2R)-2-[1-(3-chlorophenyl)-1H-1,2,3-triazol-4-yl]-N-methylpyrrolidine-1-carbimidothioate.
